SEQUENCE LISTING (22 sequences)

SEQ ID NO: 1 - protein, 304 aa, Artificial Sequence
  Mouse ADP-ribosyl Cyclase
MARGKAKEEGSWKKFIWNSEKKEFLGRTGGSWFKILLFYVIFYGCLAGIFIGTIQVMLLTISELKPTYQDRVAPPGLTQIPQIQKTEISFRPNDPKSYEAYVLNIIRFLEKYKDSAQKDDMIFEDCGNVPSEPKERGDINHERGERKVCRFKLDWLGNCSGLNDDSYGYREGKPCIIIKLNRVLGFKPKPPKNESLETYPLMMKYNPNVLPVQCTGKRDEDKDKVGNIEYFGMGGYYGFPLQYYPYYGKLLQPKYLQPLLAVQFTNLTVDTEIRVECKAYGENIGYSEKDRFQGRFDVKIEIKS

SEQ ID NO: 2 - protein, 303 aa, Artificial Sequence
  Human ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEISFRPNDPKSYEAYVLNIVRFLEKYKDSAQRDDMIFEDCGDVPSEPKERGDFNHERGERKVCRFKLEWLGNCSGLNDETYGYKEGKPCIIIKLNRVLGFKPKPPKNESLETYPVMKYNPNVLPVQCTGKRDEDKDKVGNVEYFGLGNSPGFPLQYYPYYGKLLQPKYLQPLLAVQFTNLTMDTEIRIECKAYGENIGYSEKDRFQGRFDVKIEVKS

SEQ ID NO: 3 - protein, 304 aa, Artificial Sequence
  Rat ADP-ribosyl Cyclase
MARGKAKEEGSWKKFIWNSEKKEFLGRTGGSWFKILLFYVIFYGCLAGIFIGTIQVMLLTISELKPTYQDRVAPPGLTQIPQIQKTEISFRPNDPKSYEAYVLNIIRFLEKYKDSAQKDDMIFEDCGSMPSEPKERGEFNHERGERKVCRFKLDWLGNCSGLNDESYGYKEGKPCIIIKLNRVLGFKPKPPKNESLETYPLTMKYNPNVLPVQCTGKRDEDKDKVGNIEYFGMGGFYGFPLQYYPYYGKLLQPKYLQPLLAVQFTNLTLDTEIRIECKAYGENIGYSEKDRFQGRFDVKIEVKS

SEQ ID NO: 4 - protein, 303 aa, Artificial Sequence
  Dog ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEISFRPNDPKSYEEYVRNIVRFLEKYKDSAQKDEMIFEDCGNMPSEIKERGEFNNERGERKVCRFKLEWLGNCSGINDETYGYRDGKPCVLIKLNRVLGFKPKPPKNESLEAYPVMKYNPYVLPVQCTGKRDEDKDRIGNVEYFGLGGYPGFPLQYYPYYGKLLQPKYLQPLLAVQFTNLTMDTEIRIECKAYGENIGYSEKDRFQGRFDVKIEVKS

SEQ ID NO: 5 - protein, 303 aa, Artificial Sequence
  Pig ADP-ribosyl Cyclase
MARGKAKEEGSWKKLMWNSEKKEFLGRTGGSWFKILLFYVIFYGCLAGIFIGTIQVMLLTISEFKPTYQDRVAPPGLTQIPQSQKTEISFRPNDPQSYESYVVSIVRFLEKYKDLAQKDDMIFEDCGNVPSELKERGEYNNERGERKVCRFRLEWLGNSSGLNDETYGYKDGKPCVIIKLNRVLGFKPKPPKNESLETYPVMKYNPYVLPVHCTGKRDEDKEKVGTMEYFGLGGYPGFPLQYYPYYGKLLQPKYLQPLMAVQFTNLTMDTEIRIECKAYGENIGYSEKDRFQGRFDVKIEVKS

SEQ ID NO: 6 - protein, 303 aa, Artificial Sequence
  Rabbit ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQVPQIQKTEIAFRPSDPKSYEEYVVNIVRFLEKYKDSAQKDDMVFEDCGDVPSEPKERGEFNNERGQRKVCRFKLNWLGNCSGIDDETYGYKDGKPCIIIKLNRVLGFKPKPPKNDSLEFSPGTKYNPNVLPVQCTGKRDEDKEKVGSMEYFGMGDYAGFPLQYYPYYGKLLQPKYLQPLLAVQFTNLTMDTEIRIECKAYGENIGYSEKDRFQGRFDVKIEVKS

SEQ ID NO: 7 - protein, 303 aa, Artificial Sequence
  Sheep ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEIAFRPNDPKSYMTYVDNIDNFLKKYRDSAQKDDMIFEDCGNVPSELKDRGEFNNEQGERKVCRFKLEWLGNCSGINDETYGYKEGKPCVIIKLNRVLGFKPKPPKNESLETYPVMKYNPYVLPVQCTGKRDEDKEKVGSIEYFGLGGYPGFPLQYYPYYGKLLQPKYLQPLLAVQFTNLTMDTEIRIECKAYGENIGYSEKDRFQGRFDVKIEVKS

SEQ ID NO: 8 - protein, 305 aa, Artificial Sequence
  Chicken ADP-ribosyl Cyclase
MARGKANDGDGNWKKFIWNSEKKELLGRTGGSWFKILLFYVIFYGCLAGIFIGTIQVMLLTVSEFEPKYQDRVAPPGLTQVPQVQKTEISFTVNDPKSYDPYVKNLEGFLNKYSAGEQTDNIVFQDCGDIPTDYKERGPYNDAQGQKKVCKFKREWLENCSGLQDNTFGYKDGKPCILVKLNRIIGFKPKAPENESLPSDLAGKYNPYLIPVHCVAKRDEDADKIGMVEYYGMGGYPGFALQYYPYYGRLLQPQYLQPLVAVQFTNLTYDVEVRVECKEYGQNIQYSDKDRFQGRFDIKFDIKSS

SEQ ID NO: 9 - protein, 133 aa, Artificial Sequence
  Cattle ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEIAFRPNDPKSYMTYVDNIDNFLKKYSALVDLVFSQVKTMDNKRMNT

SEQ ID NO: 10 - protein, 303 aa, Artificial Sequence
  Chimpanzee ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEISFRPNDPKSYEAYVLNIVRFLEKYKDSAQRDDMIFEDCGDVPSEPKERGDFNHERGERKVCRFKLEWLGNCSGLNDETYGYKEGKPCIIIKLNRVLGFKPKPPKNESLETYPVMKYNPNVLPVQCTGKRDEDKDKIGNVEYFGLGNSPGFPLQYYPYYGKLLQPKYLQPLLAVQFTNLTMDTEIRIECKAYGENIGYSEKDRFQGRFDVKIEVKS

SEQ ID NO: 11 - protein, 303 aa, Artificial Sequence
  Horse ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEISFRPNDPKSYEAYVLNIVRFLEKYKDSAQKDDMIFEECGSVPSELKERGEFNNERGERKVCRFKLEWLGNCSGINDETYGYKEGKPCVIIKLNRVLGFKPKPPKNESLETYPVMKYSPYVLPVQCTGKRDEDKEKIGNVEYFGLGGYPGFPLQYYPYYGKLLQPKYLQPLLAVQFTNLTMDTEIRIECKAYGENIGYSDKDRFQGRFDVKIEVKS

SEQ ID NO: 12 - protein, 304 aa, Artificial Sequence
  Frog ADP-ribosyl Cyclase
MARDKAKETDGGWRKFIWNPDKKEFLGRTGGSWFKILLFYLIFYGCLAGIFIGTIQVLLLTISEYEPKYQDRVAPPGLTQVPKAVKTEINFSPNDPDSYNDYVQSMEKFISKYSNENQVSDKFEDCGTMPGQYRERGGLNKDGGQKKSCVFRRQWLQNCSGIDDQTFGFAEGKPCVIVKLNRIVAFKPVPPQNNSLPPEMTANYNPYVIPIHCQGKRDEDIPNIREVKYYGMGGFAGFPLNYYPYYGKLLQPEYLQPLIAVQFTNLTFNTEIRIECKAYGENIDYHDKDRFQGRFDIKFDIKSS

SEQ ID NO: 13 - protein, 303 aa, Artificial Sequence
  Goat ADP-ribosyl Cyclase
MARGKAKEEGSWKKFIWNSEKKEFLGRTGGSWFKILLFYVIFYGCLAGIFIGTIQVMLLTISEFKPTYQDRVAPPGLTQIPQIQKTEIAFRPNDPKSYMTYVDNIDNFLKKYRDSAQKDDMIFEDCGNVPSELKDRGEFNNEQGERKVCRFKLEWLGNCSGINDETYGYREGKPCVIIKLNRVLGFKPKPPKNESLETYPVMKYNPYVLPVQCTGKRDEDKEKVGSIEYFGLGGYPGFPLQYYPYYGKLLQPKYLQPLLAVQFTNLTMDTEIRIECKAYGENIGYSEKDRFQGRFDVKIEVKS

SEQ ID NO: 14 - protein, 319 aa, Artificial Sequence
  Turkey ADP-ribosyl Cyclase
MTVLWKKFPNASDVTAIGFTLPGYQRVFFNSWRTALLHQALPASPGCVKILLFYVIFYGCLAGIFIGTIQVMLLTVSEFEPKYQDRVAPPGLTQVPQVQKTEISFTVSDPKSYDPYVKNLEGFLSKYSAGEQTDNIVFQDCGDVPMDYKERGPYNDDQGQKKVCKFKREWLENCSGLQDNTFGYKEGKPCILVKLNRIIGFKPKAPENESLPLGLAGKYNPFLIPVHCVAKRDEDSDKIGTVEYYGMGGYPGFALQYYPYYGKLLQPHYLQPLVAVQFTNLTYDVEVRVECRAYGQNIQYSDKDRFQGRFDIKFDIRSS

SEQ ID NO: 15 - protein, 306 aa, Artificial Sequence
  Guinea Pig ADP-ribosyl Cyclase
MARGKAKEEGSWKKFIWNSEKKEFLGRTGGSWFKILLFYVIFYGCLAGIFIGTIQVMLLTISELKPTYQDRVAPPGLTQIPQIQKTEISFRPADPKSYEAYVLNIYRFLEKYKDAAQKDDMIFEDCSTVPSEPKERGDFNHERGERKVCRFKLEWLGNCSGQNDDSYGYRDGKPCIIIKLNRVLGFKPKPPKNDSSETVEIYSTMKYNPYVLPVQCTGKREEDKDKIGSVEYFGLGGYAGFPLQYYPYYGKLLQPKYLQPLLAVQFTNLTTDTEVRIECKAYGENIGYSEKDRFQGRFDVKIEVKS

SEQ ID NO: 16 - protein, 280 aa, Artificial Sequence
  E.granulos ADP-ribosyl Cyclase
MVRCSERYRNLGLAIFNPKEKKFCGRTCRSWALIFVYYLIFYSCLAGFWIGMLSVLIFAMIDTTVPSLTGMQSLLKLNPGLGILPPVDSEGTLIQLTLFDSKQKQDYLNFMQSYLMGYSNISTNCDFENGTRINSSILEPCEFPLSLLGPCADPADYINSHNNFCFYLKLNKIYGYLPDIEGNKIPIQCGPANSFDGANLGQPVYYPSVRTANGTFGYFSSVAFPYLNQPHYQVPLLAVTFPDIKPNTVVMVSCAVLNVKDQEPFRFDLAIDMDRPIALT

SEQ ID NO: 17 - protein, 232 aa, Artificial Sequence
  S.haematob ADP-ribosyl Cyclase
MGMLIIITQLIISNDQPYITGMDSPLALSPGLGMRPRNDFKTTLIAYASSDPQTYMPFVQDIRTFLYFYEEVNIQPQDGFATCDKIKSPDDVDLVCKFYPHDMGVCVKENNFGYDRSQPCVIMKINKVYGWLPDIVNKTLSNNPLVRCHGQNPQDLENFGQVLYFPNITVDGKTYGYFSHLYFPYLMQVAYRSPLVAVQFASPKRHVLLMVRCELFNVRNPGDPMDFEILVD

SEQ ID NO: 18 - protein, 190 aa, Artificial Sequence
  T.spiralis ADP-ribosyl Cyclase
MGYQPWLRDDPESTLIYFNRSEPSTYKNNSSNRENTHIACAFDLIEHFESQGCGEKDDFGFKNATPCIVLTLNKLIGWEPVAYPKDSAPDAIKDHYNYSTPDVAIACEGEFPVDQEHIGPLQYIPPTGIPHKFFPYRVMPNYHQPFALVKFVGPPKGILIEVECKAYAYNIMHDRSYRLGMVHFELLIDS

SEQ ID NO: 19 - protein, 345 aa, Artificial Sequence
  Drosophila ADP-ribosyl Cyclase
MPEDVMVPSGNYKVRKRFRNEEHRRSKKDMPWTKQILDLNENRFFGRTAWAWCRIVSFYLLLYLLIFLILLCLYLIFRFYFIQKDRPSILKEAPGLSIVPRNESTITFYYNQMPDIYPLCDRIDEFLEKLDDEAFEYFHECNGDKLWGYNEKKPCVFVKLNKIFGFKPEVYTSPTELPSEAPPELTTIMGKFKGHDRIWLTCELSQGKLPKIVYIPGPYFDTEELAGVSRVVALQLTEMPENKEIFVSCRVWAKNIKIDLKQTGRGNAKFSMKMKVKKSNNNPPDNLETRPTKFKVWKPNQDQQRIFYDEDLQLPDVPDLEAEDDGVKTDTRSAIPTEPPDKLKR

SEQ ID NO: 20 - protein, 306 aa, Artificial Sequence
  Zebrafish Type A ADP-ribosyl Cyclase
MPANKDGDGGWKSFIWNSDKKEFLGRTGCSWLKIFIFYVIFYGCLAGIFIGTIQAMLLTLSNYKPTYQDRVAPPGLSHSPRPDKAEINYNINDESTYLPYVNHIDAFLKAYNEDVQKDDTKFEECGDKPQFYTDRGELESDNGVRKACRFRREWLGECSGQKDEKLKNYGFDDGQPCLIVKLNRIVNFMPRPPASNDSIPEAVRPKLQGNVIPIHCSSKREEEANLLGQIKYFGLGTGFPLQYYPYYGKLLQPQYLQPLVAIKFYNITTDVDVRVECKVYGENIDYSEKDRSQGRFDIKFTIKTKS

SEQ ID NO: 21 - protein, 302 aa, Artificial Sequence
  Zebrafish Type B ADP-ribosyl Cyclase
MPAQNKDDGGWKKFVWNSEKKEFLGRTGGSWAKIFLFYLIFYGCLAGIFIGTIQILLLTLSDYKPTWQDRVAPPGLTHFPRSDKSEIAINLDDEVSFLNYVKVMREFLTSYDQEKQLDNMQFENCGESPLDYKNRGDLESDVGVRRACQFSREWLGPCSGLDDPYFGFKEGKPCLIAKLNRIVNFRPKPPVSNESIPEEVQHKVQPYLIPIHCTNKKEEDAGKLGEVRYYGFGGGFPLQYYPYYGKLLHPQYLQPLVAIQFLNITPNTDMRIECKVYGENIYYHDKDRYQGRFDVKFNIKKS

SEQ ID NO: 22 - RNA, 25 nt, Artificial Sequence
  ADPRC siRNA
cccaagaaugaauccuuggagacuu

(19) United States
(12) Patent Application Publication
(10) Pub. No.: US 2022/0235343 A1
(43) Pub. Date: Jul. 28, 2022

(54) NOVEL ADP-RIBOSYL CYCLASE AND INHIBITOR THEREOF

(71) Applicants: INDUSTRIAL COOPERATION FOUNDATION CHONBUK NATIONAL UNIVERSITY, Jeollabuk-do (KR); DNT Co., Ltd., Jeollabuk-do (KR)

(72) Inventors: Uh-Hyun KIM, Jeollabuk-do (KR); Tae-Sik NAM, Jeollabuk-do (KR)

(73) Assignees: INDUSTRIAL COOPERATION FOUNDATION CHONBUK NATIONAL UNIVERSITY, Jeollabuk-do (KR); DNT Co., Ltd., Jeollabuk-do (KR)

(21) Appl. No.: 17/623,045

(22) PCT Filed: Jun. 25, 2020
(86) PCT No.: PCT/KR2020/008283

§ 371 Date: Dec. 27, 2021

(30) Foreign Application Priority Data
Jun. 27, 2019 (KR) 10-2019-0076979
Jun. 23, 2020 (KR) 10-2020-0076635

(51) Int. Cl. C12N 9/24; C12Q 1/6883; C07K 14/705; A61P 13/12

(52) U.S. Cl. CPC C12N 9/2497; C12Q 1/6883; C12Q 2600/156; A61P 13/12; C12Y 302/02006; C07K 14/705

(57) ABSTRACT

The present disclosure relates to a pharmaceutical composition containing an inhibitor against the expression or activation of a novel ADP-ribosyl cyclase or a naturally occurring variant thereof as an active ingredient for preventing or treating an ADP-ribosyl cyclase-mediated disease. In addition, the present disclosure relates to a composition for diagnosis of an ADP-ribosyl cyclase-mediated disease, the composition containing an agent for measuring a gene expression level or protein level of the ADP-ribosyl cyclase or a naturally occurring variant thereof. The composition of the present disclosure has the effect of inhibiting calcium increase in kidney cells, which is attributed to angiotensin II-induced ADP-ribosyl cyclase expression or activation, and as such can be advantageously used as a therapeutic agent for an ADP-ribosyl cyclase-mediated disease, particularly a renal disease.

### TECHNICAL FIELD

The present disclosure relates to a novel ADP-ribosyl cyclase which converts NAD⁺ to cyclic ADP-ribose and an inhibitor thereof.

### BACKGROUND ART

ADPRC is an enzyme which synthesizes cyclic ADP-ribose (cADPR) from NAD⁺ and synthesizes nicotinic acid adenine dinucleotide phosphate (NAADP) from NADP⁺. It exists widely in from plants to mammals. The ADPRC is known to regulate various cellular functions by releasing calcium from intracellular stores [Berridge M J, Bootman M D, Roderick H L. Nat Rev Mol Cell Biol. 4, 517-529, 2003; Lee H C, Mol. Med. 12, 317-323, 2006].

In stable state, the intracellular calcium concentration is maintained at a baseline of 10⁻⁷ M or lower. However, in activated state, the intracellular calcium concentration is increased up to 10 times of the baseline. In pathological state, where the calcium concentration is maintained high continuously, many cellular functions are affected and, as a result, the normal functions of cells are lost.

In hypertension or diseases accompanying hypertension such as diabetes, obesity, dementia, ischemia or cellular proliferation, the intracellular calcium concentration is maintained above a normal level due to anomaly in the regulation of intracellular calcium metabolism [Resnick L M. Am. J. Hypertens. 6: 123S-134S, 1993].

For instance, patients with chronic diseases such as hypertension show high levels of intracellular calcium concentration in vascular smooth muscle because of various hereditary, environmental or secondary causes of some diseases. As a result, peripheral vascular resistance is increased due to the contraction of the vascular smooth muscle, which causes hypertension by maintaining blood pressure consistently higher than the normal level.

It is known that the increased blood pressure secondarily causes the proliferation and hypertrophy of vascular smooth muscle cells and fibrous tissues, which further increases peripheral vascular resistance and blood pressure, leading to myocardial infarction, angina, palsy, chronic heart failure, and even death due to the dysfunction of the cardiovascular system, brain, kidney, etc. [Cowley A W Jr. Physiol Rev. 72: 231-300, 1992].

It is known that the calcium increase by the activation of CD38, which is a type of ADPRC, i.e., cADPR, plays an important role in insulin secretion from the pancreas. In addition, the induction of diabetes increases the production of angiotensin. It is well known that this peptide hormone not only induces renal diseases, hypertension and heart diseases but also activates ADPRC.

Existing in immune cells, cardiomyocytes, pancreatic beta cells, extensor muscle cells, neurons, etc., ADPRC increases intracellular calcium concentration and regulates several physiological activities in association with the receptors of several hormones. The dysregulation of the expression or activation of ADPRC may cause abnormalities in the regulation of physiological phenomena (immunity, renal function, insulin secretion and cardiovascular function).

ADPRC is activated by several hormones. This enzyme produces cADPR from the substrate NAD⁺. The cADPR increases intracellular calcium level in almost all organs. In addition, abnormal increase in calcium owing to activation of ADPRC has been known as the cause of various pathologies such as insulin secretion [Kim B J, Park K H, Yim C Y, Takasawa S, Okamoto H, Im M J, Kim U H. Diabetes. 57: 868-878, 2008], cellular hypertrophy [Gul R, Park J H, Kim S Y, Jang K Y, Chea J K, Ko J K, Kim U H. Cardiovasc Res. 81: 582-591, 2009], cell proliferation [Kim S Y, Gul R, Rah S Y, Kim S H, Park S K, Im M J, Kwon H J, Kim U H. Am J Physiol Renal Physiol. 294: F989-F989, 2008], etc.

The ADPRC that has been studied best thus far is the T-cell surface antigen CD38. This molecule is expressed widely not only in immune cells but also in many organs.

However, it has been found out that ADPRCs different from CD38 are expressed in the heart, kidney and brain [Partida-Sanchez S, Cockayne D A, Monard S, Jacobson E L, Oppenheimer N, Garvy B, Kusser K, Goodrich S, Howard M, Harmsen A, Randall T D, Lund F E. Nat Med 7: 1209-16, 2001].

Accordingly, the regulation of tissue-specific ADPRC expression will be helpful in treating a disease induced by increased intracellular calcium level. The activation mechanism of ADPRC is not known well yet.

The foregoing description in the Background section is only for enhancing the understanding about the background of the present disclosure, and it should not be regarded as admitting that it is well known to those having ordinary knowledge in the art.

### DISCLOSURE

### Technical Problem

The inventors of the present disclosure have found a new type of ADPRC other than the existing ADPRCs known in mammals (CD38 and CD157), identified its activity and completed the present disclosure.

The present disclosure is directed to providing a novel ADP-ribosyl cyclase (ADPRC) or a naturally occurring variant thereof.

The present disclosure is also directed to providing a nucleic acid molecule which encodes the ADP-ribosyl cyclase or a naturally occurring variant thereof.

The present disclosure is also directed to providing a vector including the nucleic acid molecule.

The present disclosure is also directed to providing a host cell including the vector.

The present disclosure is also directed to providing a catalyst composition converting NAD⁺ to cyclic ADP-ribose (cADPR), which contains the ADP-ribosyl cyclase or a naturally occurring variant thereof, a nucleic acid molecule encoding the same or a vector including the nucleic acid molecule.

The present disclosure is also directed to providing a pharmaceutical composition for preventing or treating an ADP-ribosyl cyclase-mediated disease, which contains an inhibitor against the expression or activation of the novel ADP-ribosyl cyclase or a naturally occurring variant thereof as an active ingredient.

The present disclosure is also directed to providing a food composition for preventing or alleviating an ADP-ribosyl cyclase-mediated disease, which contains an inhibitor against the expression or activation of the novel ADP-ribosyl cyclase or a naturally occurring variant thereof as an active ingredient.

The present disclosure is also directed to providing an animal model wherein a hetero-type gene of the ADP-ribosyl cyclase or a naturally occurring variant thereof is deleted.

The present disclosure is also directed to providing a method for providing information for diagnosis of an ADP-ribosyl cyclase-mediated disease.

The present disclosure is also directed to providing a composition for diagnosis of an ADP-ribosyl cyclase-mediated disease, which contains an agent for measuring a gene expression level or protein level of the ADP-ribosyl cyclase or a naturally occurring variant thereof.

The present disclosure is also directed to providing a kit for diagnosis of an ADP-ribosyl cyclase-mediated disease, which includes an agent for measuring a gene expression level or protein level of the ADP-ribosyl cyclase or a naturally occurring variant thereof.

The present disclosure is also directed to providing a method for screening a substance for preventing or treating the ADP-ribosyl cyclase-mediated disease.

Other purposes and advantages of the present disclosure will become more apparent by the following detailed description, claims and drawings.

### Technical Solution

In an aspect, the present disclosure provides an ADP-ribosyl cyclase (ADPRC) including an amino acid sequence of SEQ ID NO 1 or a naturally occurring variant thereof.

The inventors of the present disclosure have made consistent efforts to discover a novel ADP-ribosyl cyclase other than the previously known ADP-ribosyl cyclases, as an enzyme which converts NAD⁺ to cyclic ADP-ribose (cADPR), and have completed the present disclosure as a result.

In the present specification, the term “ADP-ribosyl cyclase” or “ADP-ribosyl cyclase (ADPRC)” refers to an enzyme which converts NAD⁺ to cyclic ADP-ribose (cADPR).

In the present specification, the term “variant of ADP-ribosyl cyclase” refers to a variant which is obtained from natural or artificial substitution, deletion or addition of a part of the amino acid sequence of the ADP-ribosyl cyclase and retains the catalytic activity of the ADP-ribosyl cyclase of converting NAD⁺ to cyclic ADP-ribose (cADPR).

In the present specification, the term “naturally occurring variant of ADP-ribosyl cyclase” refers to a naturally occurring variant of the ADP-ribosyl cyclase including the amino acid sequence of SEQ ID NO 1, which retains the catalytic activity of converting NAD⁺ to cyclic ADP-ribose (cADPR).

In a specific exemplary embodiment of the present disclosure, the naturally occurring variant of ADP-ribosyl cyclase of the present disclosure is a naturally occurring variant selected from a group consisting of an interspecies variant, a species homolog, an isoform, an allelic variant, a conformational variant, a splice variant and a point mutation variant.

The naturally occurring variant of ADP-ribosyl cyclase of the present disclosure has specifically 50% or higher homology, more specifically 60% or higher homology, further more specifically 70% or higher homology, even more specifically 80% or higher homology, most specifically 90% or higher homology, to ADP-ribosyl cyclase including the amino acid sequence of SEQ ID NO 1.

In a specific exemplary embodiment of the present disclosure, the naturally occurring variant of ADP-ribosyl cyclase of the present disclosure originates from an organism selected from a group consisting of mammals, birds, reptiles, amphibians and fish.

In an example of the present disclosure, it was confirmed that the naturally occurring variant of ADP-ribosyl cyclase of the present disclosure has 70% or higher homology for mammals, 60% or higher homology for birds, reptiles and amphibians and 50% or higher homology for fish, to the ADP-ribosyl cyclase including the amino acid sequence of SEQ ID NO 1.

In an example of the present disclosure, it was confirmed that the naturally occurring variant of ADP-ribosyl cyclase of the present disclosure is an enzyme exhibiting a very high proportion of interspecies conserved sequences with 96% homology for human and rat, 93% homology for chimpanzee, 91% homology for guinea pig and horse, 90% homology for dog, goat and sheep, 89% homology for rabbit, 88% homology for pig, 78% homology for cattle, 70% homology for chicken, 63% homology for frog and 62% homology for turkey, to the ADP-ribosyl cyclase including the amino acid sequence of SEQ ID NO 1 (FIG. 4).

In a specific exemplary embodiment of the present disclosure, the naturally occurring variant of ADP-ribosyl cyclase of the present disclosure includes an amino acid sequence selected from a group consisting of SEQ ID NOS 2-21.

In another aspect, the present disclosure provides a nucleic acid molecule encoding the ADP-ribosyl cyclase or naturally occurring variant thereof, a vector including the nucleic acid molecule or a host cell including the vector.

The nucleic acid molecule of the present disclosure may be an isolated or recombinant nucleic acid molecule, and includes not only a single-stranded or double-stranded DNA or RNA but also a sequence complementary thereto. When the “isolated nucleic acid” is a nucleic acid isolated from a natural origin, the nucleic acid is a nucleic acid separated from nearby gene sequences existing in the isolated genome of an individual. For nucleic acids, e.g., PCR products, cDNA molecules or oligonucleotides, synthesized enzymatically or chemically from a template, the nucleic acids produced from these procedures may be understood as isolated nucleic acid molecules. An isolated nucleic acid molecule may be a component of a fragment or a larger nucleic acid construct. A nucleic acid is “operably linked” when it is placed into a functional relationship with another nucleic acid sequence. For example, a DNA for a presequence or a secretory leader is operably linked to a DNA for a polypeptide if it is expressed as a preprotein that participates in the secretion of the polypeptide. A promoter or an enhancer is operably linked to a coding sequence if it affects the transcription of the polypeptide sequence. And, a ribosome-binding site is operably linked to a coding sequence if it is positioned to facilitate translation. In general, “operably linked” means that the DNA sequences being linked are contiguous and, in the case of a secretory leader, contiguous and present in the same reading frame. However, enhancers do not have to be contiguous. The linking is accomplished by ligation at convenient restriction enzyme sites. If such sites do not exist, synthetic oligonucleotide adaptors or linkers are used in accordance with common methods.

In a specific exemplary embodiment of the present disclosure, the nucleic acid molecule is a cDNA.

In the present specification, the term “vector” refers to a carrier capable of inserting a nucleic acid sequence for introduction into a cell capable of replicating the nucleic acid sequence. The nucleic acid sequence may be exogenous or heterologous. The vector may be a plasmid, a cosmid or a virus (e.g., a bacteriophage), although not being limited thereto. Those skilled in the art can construct the vector according to the standard recombination technology (Maniatis, et al., Molecular Cloning, A Laboratory Manual, Cold Spring Harbor Press, Cold Spring Harbor, N.Y., 1988; Ausubel et al., In: Current Protocols in Molecular Biology, John, Wiley & Sons, Inc., NY, 1994, etc.).

In the present specification, the term “expression vector” refers to a vector including a nucleic acid sequence encoding at least a part of a transcribed gene product. In some cases, an RNA molecule is post-translated into a protein, a polypeptide or a peptide. The expression vector may contain various regulatory sequences. A vector or an expression vector may further contain a nucleic acid sequence that provides another function, in addition to a regulatory sequence regulating transcription and translation.

In the present specification, the term “host cell” refers to a cell of any transformable organism, including a eukaryote and a prokaryote, which is capable of replicating the vector or expressing a gene encoded by the vector. The host cell may be transfected or transformed by the vector, which means a process by which an exogenous nucleic acid molecule is delivered or introduced into the host cell.

The host cell of the present disclosure may be specifically a bacterial cell, a yeast cell, an animal cell or a human cell (CHO cell, HeLa cell, HEK293 cell, MES13 cell, BHK-21 cell, COS7 cell, COP5 cell, A549 cell, NIH3T3 cell, etc.), although not being limited thereto.

In another aspect, the present disclosure provides a catalyst composition which converts NAD⁺ to cyclic ADP-ribose (cADPR), which contains the ADP-ribosyl cyclase or naturally occurring variant thereof, the nucleic acid molecule encoding the same or the vector including the nucleic acid molecule.

The catalyst composition of the present disclosure effectively catalyzes the process of converting NAD⁺ to cADPR.

In another aspect, the present disclosure provides a composition containing an inhibitor against the expression or activation of the novel ADP-ribosyl cyclase or a naturally occurring variant thereof.

In a specific exemplary embodiment of the present disclosure, the composition of the present disclosure is a pharmaceutical composition for preventing or treating an ADP-ribosyl cyclase-mediated disease.

The pharmaceutical composition of the present disclosure may contain: (a) the inhibitor against the expression or activation; and (b) a pharmaceutically acceptable carrier.

In a specific exemplary embodiment of the present disclosure, the composition of the present disclosure is a food composition for preventing or alleviating an ADP-ribosyl cyclase-mediated disease.

In another aspect, the present disclosure provides a method for preventing or treating an ADP-ribosyl cyclase-mediated disease, which includes a step of administering a pharmaceutically effective amount of the pharmaceutical composition to a subject.

In another aspect, the present disclosure provides an inhibitor against the expression or activation of the novel ADP-ribosyl cyclase or a naturally occurring variant thereof for use in therapy.

The ADP-ribosyl cyclase-mediated disease to be prevented or treated is not limited specially. The disease may be specifically diabetes or renal disease, more specifically renal failure, nephropathy, nephritis, renal fibrosis or nephrosclerosis.

In a specific exemplary embodiment of the present disclosure, the renal failure may be chronic renal failure, acute renal failure or mild renal failure before dialysis.

In a specific exemplary embodiment of the present disclosure, the nephropathy may be nephropathy syndrome, lipoid nephropathy, diabetic nephropathy, immunoglobulin A (IgA) nephropathy, analgesic nephropathy or hypertensive nephropathy.

In an example of the present disclosure, it was confirmed that the composition of the present disclosure is a good candidate as a therapeutic agent for renal diseases including chronic renal failure or diabetic nephropathy through significant decrease in kidney to body weight, significant increase in creatinine clearance rate and significant decrease in urine albumin (FIG. 6). In addition, it was confirmed that it lowers the activity of ADP-ribosyl cyclase and the concentration of cADPR to normal levels (FIG. 7), lowers the expression levels of TGF-β1, fibronectin and collagen IV to normal levels (FIG. 8), and recovers the histopathological change of the kidney by recovering glomerulus hypertrophy, infiltration of inflammatory cells and formation of transitional epithelial cells to normal levels (FIG. 9). Furthermore, it was confirmed that it can be applied to hypertensive nephropathy by significantly increasing creatinine clearance rate in a hypertension model (FIG. 11B). Therefore, the composition can be used as a therapeutic agent for various renal diseases caused by the failure or loss of kidney function.

In the present specification, the term “inhibitor of expression” refers to a substance which inhibits the expression of the ADP-ribosyl cyclase, and it may be easily prepared by those having ordinary skill in consideration of the structure and function of the ADP-ribosyl cyclase.

The inhibitor of expression of the present disclosure is specifically any one selected from a group consisting of an antisense oligonucleotide, a siRNA, a shRNA, a miRNA, a ribozyme, a DNAzyme and a PNA (protein nucleic acid) binding complementarily to the mRNA of the ADP-ribosyl cyclase or naturally occurring variant thereof, although not being limited thereto.

In a specific exemplary embodiment of the present disclosure, the siRNA includes a nucleotide sequence of SEQ ID NO 22.

In the present specification, the term “inhibitor of activation” refers to a substance which inhibits the activation of the expressed ADP-ribosyl cyclase protein, and it is specifically any one selected from a group consisting of a compound, a peptide, a peptide mimetic, an aptamer and an antibody binding specifically to the ADP-ribosyl cyclase or naturally occurring variant thereof protein.

In a specific exemplary embodiment of the present disclosure, the compound is selected from a group consisting of 4,4′-dihydroxyazobenzene, 2-(1,3-benzoxazol-2-ylamino)-1-methylquinazolin-4(1H)-one and dicaffeoylquinic acid (DCQA).

Specifically, the 4,4′-dihydroxyazobenzene of the present disclosure may be represented by Chemical Formula 1:

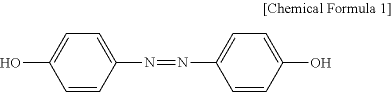 [SMILES: Oc1ccc(N=Nc2ccc(O)cc2)cc1]

Specifically, the 2-(1,3-benzoxazol-2-ylamino)-1-methylquinazolin-4(1H)-one of the present disclosure may be represented by Chemical Formula 2:

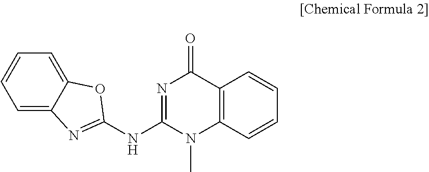 [SMILES: Cn1c(Nc2nc3ccccc3o2)nc(=O)c2ccccc21]

The dicaffeoylquinic acid (DCQA) of the present disclosure may be any one selected from a group consisting of the compounds represented by Chemical Formulas 3-8 (1,4-DCQA, 3,4-DCQA, 3,5-DCQA, 4,5-DCQA, 1,3-DCQA and 1,5-DCQA):

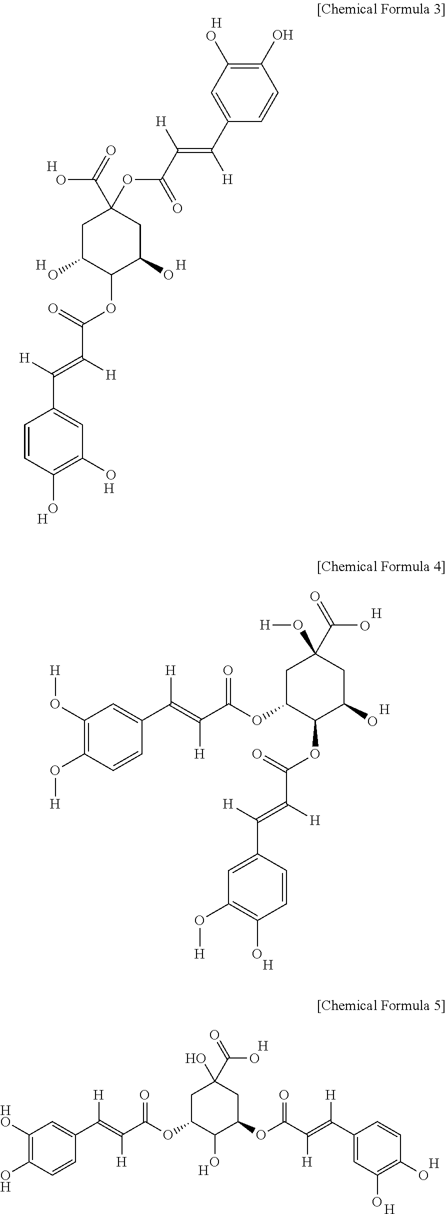

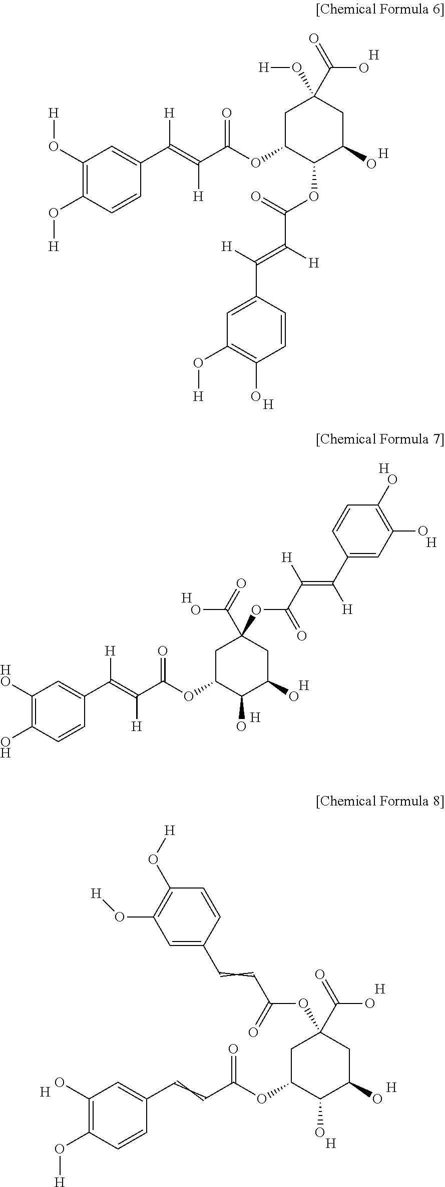

In an example of the present disclosure, it was confirmed that the inhibitor against the expression or activation of the novel ADP-ribosyl cyclase according to the present disclosure contributes to the inhibition of a renal disease via a mechanism of regulating the expression or activation of ADPRC.

Accordingly, the inhibitor against the expression or activation of the novel ADP-ribosyl cyclase of the present disclosure may be used as a clinically useful selective inhibitor for individual cells and, furthermore, as an agent for preventing, alleviating and/or treating a renal disease.

The composition of the present disclosure may contain, in addition to the inhibitor against the expression or activation of the novel ADPRC as an active ingredient, other previously known therapeutic agents for ADPRC-related diseases.

The pharmaceutical composition of the present disclosure may be formulated into a suitable form together with a pharmaceutically acceptable carrier. ‘Pharmaceutically acceptable’ means that the carrier which is physiologically acceptable and does not cause allergic reactions such as gastrointestinal disturbance, vertigo, etc. and similar responses.

The pharmaceutically acceptable carrier contained in the pharmaceutical composition of the present disclosure includes, as those commonly used in formulation, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., although not being limited thereto. In addition to the above-described ingredients, the pharmaceutical composition of the present disclosure may further contain a lubricant, a wetting agent, a sweetener, a flavorant, an emulsifier, a suspending agent, a preservative, etc. Suitable pharmaceutically acceptable carriers and formulations are described in detail in Remington's Pharmaceutical Sciences (19th ed., 1995).

The pharmaceutical composition of the present disclosure may be administered orally or parenterally. Specifically, it may be administered parenterally, e.g., intravenously, topically, intraperitoneally, etc.

An appropriate administration dosage of the pharmaceutical composition of the present disclosure varies depending on such factors as formulation method, administration method, the age, body weight, sex, pathological condition and diet of a patient, administration time, administration route, excretion rate and response sensitivity, and an administration dosage effective for the desired treatment or prevention may be easily determined by an ordinarily skilled physician. In a specific exemplary embodiment of the present disclosure, a daily administration dosage of the pharmaceutical composition of the present disclosure is 0.0001-100 mg/kg.

The pharmaceutical composition of the present disclosure may be prepared as a single-dose or multiple-dose formulation using a pharmaceutically acceptable carrier and/or excipient according to a method that can be easily carried out by those having ordinary knowledge in the art. The formulation may be in the form of a solution in an oily or aqueous medium, a suspension, an emulsion, an extract, a powder, a granule, a tablet or a capsule, and may further contain a dispersant or a stabilizer.

When the composition of the present disclosure is prepared as a food composition, it may further contain ingredients commonly added for preparation of food in addition to the inhibitor against the expression or activation of the ADP-ribosyl cyclase as an active ingredient. For example, it may contain a protein, a carbohydrate, a fat, a nutrient, a condiment and a flavorant. The examples of the carbohydrate may include common sugars such as a monosaccharide, e.g., glucose, fructose, etc., a disaccharide, e.g., maltose, sucrose, oligosaccharides, etc., a polysaccharide, e.g., dextrin, cyclodextrin, etc., and sugar alcohols such as xylitol, sorbitol, erythritol, etc. As the flavorant, a natural flavorant (thaumatin, stevia extract [e.g., rebaudioside A, glycyrrhizin, etc.]) or a synthetic flavorant (saccharine, aspartame, etc.) may be used.

For example, when the food composition of the present disclosure is prepared as a drink, citric acid, fructose syrup, sugar, glucose, acetic acid, malic acid, fruit juice, eucommiae cortex extract, jujube extract, licorice extract, etc. may be further added in addition to the inhibitor against the expression or activation of ADP-ribosyl cyclase of the present disclosure.

The food composition of the present disclosure exhibits very superior effect in alleviating renal diseases through regulation of various kidney-related pathological factors (significant decrease in kidney to body weight, significant increase in creatinine clearance rate, significant decrease in urine albumin level, decrease in expression levels of TGF-β1, fibronectin and collagen IV to normal levels, and recovery of glomerulus hypertrophy, infiltration of inflammatory cells and formation of transitional epithelial cells to normal levels).

In another aspect, the present disclosure provides an animal model wherein a hetero-type gene of ADP-ribosyl cyclase (ADPRC) or a naturally occurring variant thereof is deleted.

In a specific exemplary embodiment of the present disclosure, the animal may be a non-human mammal, a bird, a reptile, an amphibian or a fish.

In another aspect, the present disclosure provides a method for identifying an ADP-ribosyl cyclase-mediated disease, which includes: (a) a step of inducing a specific disease in the animal model wherein a hetero-type gene of ADP-ribosyl cyclase (ADPRC) or a naturally occurring variant thereof is deleted and a wild-type animal model; and (b) a step of identifying the difference between the animal models.

By using the animal models, it is possible to identify whether a specific disease (e.g., diabetes, renal disease, hypertension, obesity, etc.) has occurred as being mediated by ADP-ribosyl cyclase or regardless of ADP-ribosyl cyclase based on the difference between the animal models (e.g., difference in body weight, kidney weight, blood pressure, creatinine clearance rate, blood sugar, inflammation, degree of glomerulus hypertrophy, etc.).

In another aspect, the present disclosure provides a method for providing information for diagnosis of an ADP-ribosyl cyclase-mediated disease, which includes:

1) a step of measuring the expression or activation level of an ADP-ribosyl cyclase including an amino acid sequence of SEQ ID NO 1 or a naturally occurring variant thereof in a sample isolated from a subject; and

2) a step of determining a risk of the ADP-ribosyl cyclase-mediated disease of the subject by comparing the expression or activation level of the ADP-ribosyl cyclase or naturally occurring variant thereof in the step 1) with a normal control group.

In the present specification, the term “diagnosis” refers to identification of the presence or characteristics of a pathological condition. In the purpose of the present disclosure, the diagnosis means the identification of the occurrence or the risk of occurrence of an ADP-ribosyl cyclase-related or mediated disease.

ADPRC is activated by several hormones. This enzyme produces cADPR from the substrate NAD⁺. The cADPR increases intracellular calcium level in almost all organs. In addition, abnormal increase in calcium owing to activation of ADPRC has been known as the cause of various pathologies such as insulin secretion, cellular hypertrophy, cell proliferation, etc. Accordingly, it is possible to provide useful information for diagnosis of the ADP-ribosyl cyclase-mediated disease induced by increased intracellular calcium level based on the measurement of the expression or activation level of the ADP-ribosyl cyclase.

In another aspect, the present disclosure provides a composition for diagnosis of an ADP-ribosyl cyclase-mediated disease, which contains an agent for measuring the gene expression level or protein level of an ADP-ribosyl cyclase including an amino acid sequence of SEQ ID NO 1 or a naturally occurring variant thereof.

In a specific exemplary embodiment of the present disclosure, the agent for measuring the gene expression level is a primer or a probe binding specifically to a gene encoding the ADP-ribosyl cyclase or naturally occurring variant thereof.

In a specific exemplary embodiment of the present disclosure, the agent for measuring the protein level is an antibody binding specifically to the ADP-ribosyl cyclase or naturally occurring variant thereof or an antigen-binding fragment thereof.

Since the antibody or antigen-binding fragment binds specifically to the ADP-ribosyl cyclase or naturally occurring variant thereof, it can be used to accurately measure the amount of the ADP-ribosyl cyclase or naturally occurring variant thereof contained in a sample.

The composition for diagnosis of the present disclosure may be used to quantify the amount of the ADP-ribosyl cyclase or naturally occurring variant thereof by analyzing an antigen for the antibody based on antigen-antibody binding reaction. Specifically, the analysis based on antigen-antibody binding reaction is selected from a group consisting of ELISA (enzyme-linked immunosorbent assay), RIA (radioimmunoassay), sandwich assay, western blot polyacrylamide gel, immunoblot assay and immunohistochemical staining, although not being limited thereto.

As a substrate for antigen-antibody binding reaction, one selected from a group consisting of a nitrocellulose membrane, a PVDF membrane, a well plate synthesized from a polyvinyl resin or a polystyrene resin and a slide glass may be used, although not being limited thereto.

Specifically, a secondary antibody may be labeled with a common color developing agent. A label selected from a group consisting of HRP (horseradish peroxidase), alkaline phosphatase, colloidal gold, a fluorescein such as FITC (poly-L-lysine fluorescein isothiocyanate), RITC (rhodamine B isothiocyanate), etc. and a dye may be used. Specifically, as a substrate inducing color development, any one selected from a group consisting of TMB (3,3′,5,5′-tetramethylbezidine), ABTS [2,2′-azino-bis(3-ethylbenzothiazoline-6-sulfonic acid)] and OPD (o-phenylenediamine) may be used, although not being limited thereto.

In another aspect, the present disclosure provides a kit for diagnosis of an ADP-ribosyl cyclase-mediated disease, which includes an agent for measuring the gene expression level or protein level of the ADP-ribosyl cyclase including an amino acid sequence of SEQ ID NO 1 or a naturally occurring variant thereof.

The diagnostic kit of the present disclosure may further include one or more composition, solution or device suitable for analysis and an instruction thereabout.

In another aspect, the present disclosure provides a method for screening a substance for preventing or treating an ADP-ribosyl cyclase-mediated disease, which includes:

1) a step of treating a cell expressing an ADP-ribosyl cyclase including an amino acid sequence of SEQ ID NO 1 or a naturally occurring variant thereof with a test substance;

2) a step of measuring the gene expression level or protein level of the ADP-ribosyl cyclase or naturally occurring variant thereof as a result of treating with the test substance; and

3) a step of screening the test substance as a substance for preventing or treating an ADP-ribosyl cyclase-mediated disease if the gene expression level or protein level is decreased as compared to a control group not treated with the test substance.

Specifically, the gene expression level of the ADP-ribosyl cyclase or naturally occurring variant thereof may be measured by one or method selected from a group consisting of immunoprecipitation, radioimmunoassay (RIA), enzyme-linked immunosorbent assay (ELISA), immunohistochemistry, RT-PCR, western blot and fluorescence-activated cell sorting (FACS), although not being limited thereto.

Specifically, the protein level of the ADP-ribosyl cyclase or naturally occurring variant thereof may be measured by one or more method selected from a group consisting of SDS-PAGE, immunofluorescence, enzyme-linked immunosorbent assay (ELISA), mass spectrometry and protein chip assay, although not being limited thereto.

### Advantageous Effects

The features and advantages of the present disclosure may be summarized as follows:

(i) The present disclosure provides a pharmaceutical composition for preventing or treating an ADP-ribosyl cyclase-mediated disease, which contains an inhibitor against the expression or activation of a novel ADP-ribosyl cyclase or a naturally occurring variant thereof as an active ingredient.

(ii) The present disclosure also provides a composition for diagnosis of an ADP-ribosyl cyclase-mediated disease, which contains an agent for measuring a gene expression level or protein level of the ADP-ribosyl cyclase or a naturally occurring variant thereof.

(iii) The composition of the present disclosure may be usefully used as a therapeutic agent for an ADP-ribosyl cyclase-mediated disease, particularly a renal disease, because it is effective in inhibiting calcium increase in kidney cells by increasing the expression or activation of the novel ADP-ribosyl cyclase when stimulated with angiotensin II.

### BRIEF DESCRIPTION OF DRAWINGS

FIG. 1 shows a result of measuring the NAD glycohydrolase (NADase) activity of new ADPRC in HEK293 cells and MES13 cells.

FIG. 2 shows a result of evaluating the cADPR synthesis capability of purified FLAG-ADPRC.

FIG. 3 shows a result of measuring the intracellular cADPR production by new ADPRC when MES13 cells are stimulated with angiotensin II.

FIG. 4 shows a result of comparing the interspecies sequence homology of ADP-ribosyl cyclase of the present disclosure (human (96%), rat (96%), dog (90%), pig (88%), rabbit (89%), sheep (90%), chicken (70%), cattle (78%), chimpanzee (93%), horse (91%), frog (63%), goat (90%), turkey (62%), guinea pig (91%), Echinococcus granulosus (27%), Schistosoma haematobium (22%), Trichinella spiralis (22%), Drosophila (19%) and zebrafish (56%) as compared to mouse).

FIG. 5 shows a result of measuring the effect of new inhibitors inhibiting the NAD glycohydrolase (NADase) activity of new ADPRC.

FIG. 6 shows a result of measuring the effect of dicaffeolylquinic acid (DCQA) on the blood glucose (FIG. 6A), kidney-to-body weight ratio (FIG. 6B), creatinine clearance rate (FIG. 6C) and urine albumin level (FIG. 6D) of a diabetic renal disease mouse model.

FIG. 7 shows a result of measuring the effect of DCQA on the ADPRC activity (FIG. 7A) and cADPR concentration (FIG. 7B) in the kidney tissue of a diabetic renal disease mouse model.

FIG. 8 shows a result of measuring the effect of DCQA on the change in expression of TGF-β1, fibronectin and collagen IV in the kidney tissue of a diabetic renal disease mouse model.

FIG. 9 shows a result of observing the histopathological change in the kidney tissue of a diabetic renal disease mouse model by hematoxylin and eosin (H&E) staining.

FIG. 10 shows a result of measuring the blood glucose (FIG. 10A), kidney-to-body weight ratio (FIG. 10B) and creatinine clearance rate (FIG. 10C) in normal mouse and a diabetic renal disease model of ADPRC hetero (ADPRC (+/−)) mouse.

FIG. 11 shows a result of measuring the effect of DCQA on the blood pressure (FIG. 11A) and creatinine clearance rate (FIG. 11B) in normal mouse and a hypertension mouse model.

### BEST MODE

Hereinafter, the present disclosure is described in more detail through examples. These examples are only for illustrating the present disclosure more specifically and it will be obvious to those having ordinary knowledge in the art that the scope of the present disclosure is not limited by the examples.

### Examples

### Example 1. NAD Glycohydrolase (NADase) Activity of New ADPRC

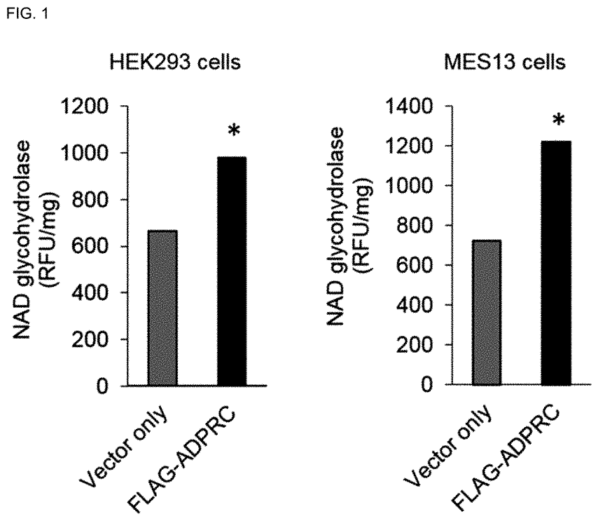

In order to investigate the ADP-ribosyl cyclase (ADPRC) activity of SEQ ID NO 1, a FLAG-ADPRC plasmid was prepared by ligating a cDNA sequence encoding ADPRC into a FLAG-CMV-2 vector and the overexpression of new ADPRC was induced by treating HEK293 cells or MES13 cells using a transfection reagent. The overexpressed new ADPRC was lysed with a lysis buffer. 45 μL of the lysed sample was treated with 5 μL of 2 mM ε-NAD (nicotinamide 1,N6-ethenoadenine dinucleotide) and incubated at 37° C. for 1 hour. Then, the enzyme-substrate reaction was stopped by treating with 50 μL of 10% trichloroacetic acid. After centrifuging for 10 minutes and adding 80 μL of the supernatant to 720 μL of 0.1 M sodium phosphate buffer, absorbance was measured at 297 nm (excitation) and 410 nm (emission) with a fluorescence spectrometer. The result is shown in FIG. 1.

### Example 2. Evaluation of cADPR Synthesis Capability of New ADPRC

The cADPR synthesis capability of ADPRC was evaluated by the method reported by Graeff R et al. [Graeff R, Lee H C. Biochem. J. 361: 379-384, 2002].

Specifically, the FLAG-ADPRC plasmid was overexpressed in HEK293 cells using a transfection reagent and then lysed with a lysis buffer. The overexpressed FLAG-ADPRC was purified from the lysed sample using a FLAG-agarose column. The purified sample was treated with 100 μM β-NAD and incubated at 37° C. for 1 hour. Then, after extracting cADPR by treating with trichloroacetic acid to a final concentration of 0.6 M, 0.1 mL of the extract or 0.1 mL of a standard cADPR solution was reacted at room temperature for 30 minutes after adding 50 μL of a mixture solution of ADPR cyclase (0.3 μg/mL), nicotinamide (30 mM) and sodium phosphate (100 mM).

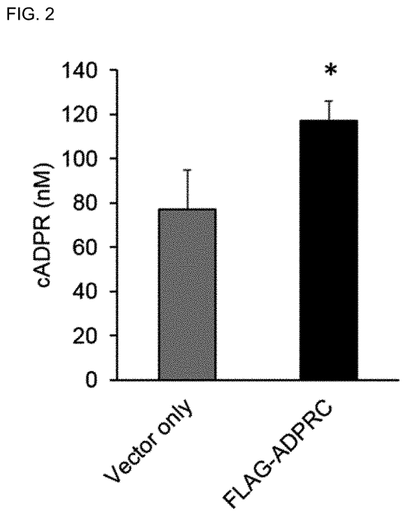

After adding ethanol (2%), alcohol dehydrogenase (100 μg/mL), resazurin (20 μM), diaphorase (10 μg/mL), FMN (10 μM), nicotinamide (10 mM), bovine serum albumin (BSA, 0.1 mg/mL) and sodium phosphate (100 mM) to the mixture solution, reaction was conducted for 2-4 hours. Then, absorbance was measured between 544 nm and 590 nm using a fluorescence spectrophotometer. The result is shown in FIG. 2.

### Example 3. Change in Intracellular cADPR Concentration in MES13 Cells by ADPRC

The change in intracellular cADPR concentration by ADPRC was investigated by the method reported by Graeff R et al. [Graeff R, Lee H C. Biochem. J. 361: 379-384, 2002].

Specifically, the expression of the new ADPRC in MES13 cells was inhibited by using a small interfering RNA (SEQ ID NO 22) as a transfection reagent. After treating the ADPRC expression-inhibited cells with 150 nM angiotensin II for 60 seconds and then extracting cADPR with 0.6 M trichloroacetic acid, 0.1 mL of the extract or 0.1 mL of a standard cADPR solution was reacted at room temperature for 30 minutes after adding 50 μL of a mixture solution of ADPR cyclase (0.3 μg/mL), nicotinamide (30 mM) and sodium phosphate (100 mM).

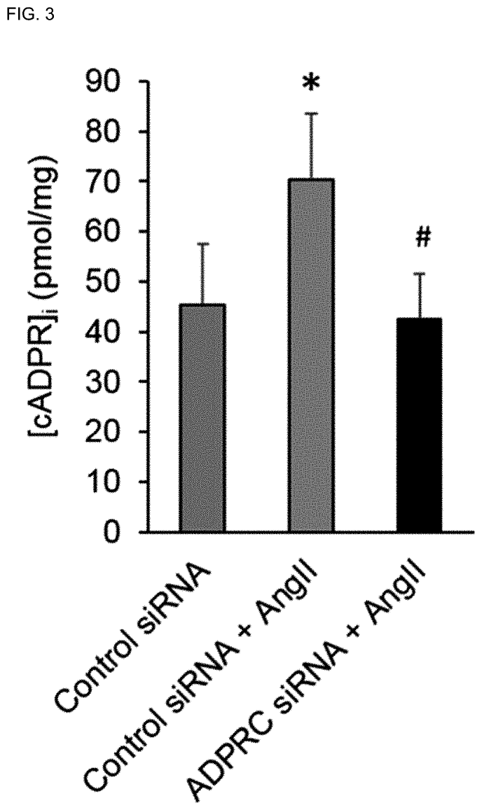

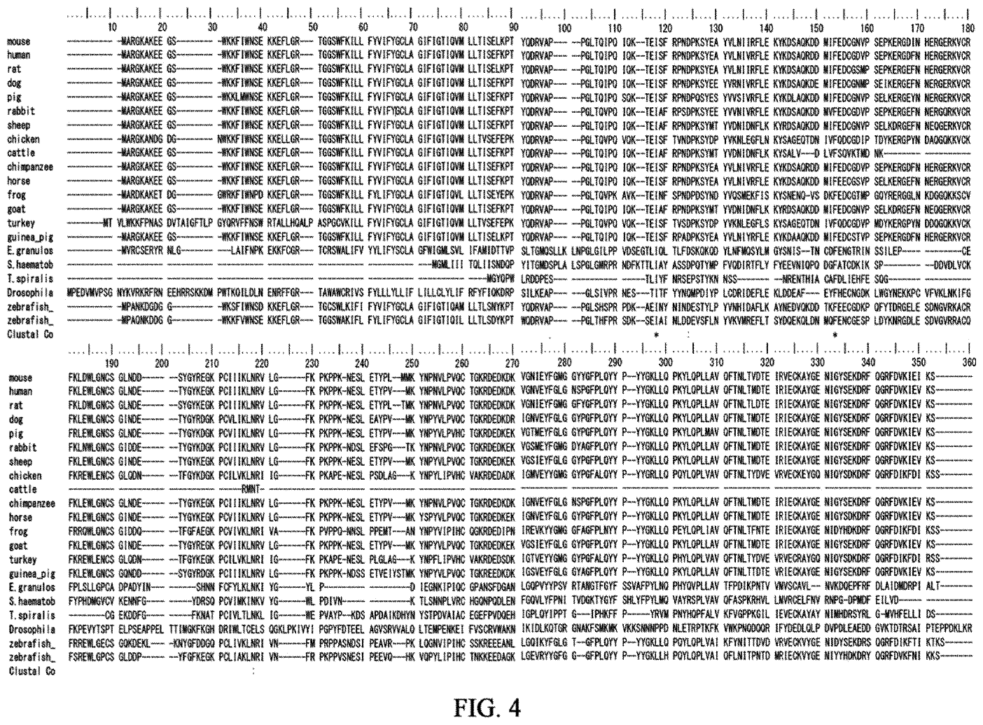

After adding ethanol (2%), alcohol dehydrogenase (100 μg/mL), resazurin (20 μM), diaphorase (10 μg/mL), FMN (10 μM), nicotinamide (10 mM), bovine serum albumin (BSA, 0.1 mg/mL) and sodium phosphate (100 mM) to the mixture solution, reaction was conducted for 2-4 hours. Then, absorbance was measured between 544 nm and 590 nm using a fluorescence spectrophotometer. The result is shown in FIG. 3.

### Example 4. Effect of New Inhibitor Inhibiting NAD Glycohydrolase (NADase) Activity of New ADPRC

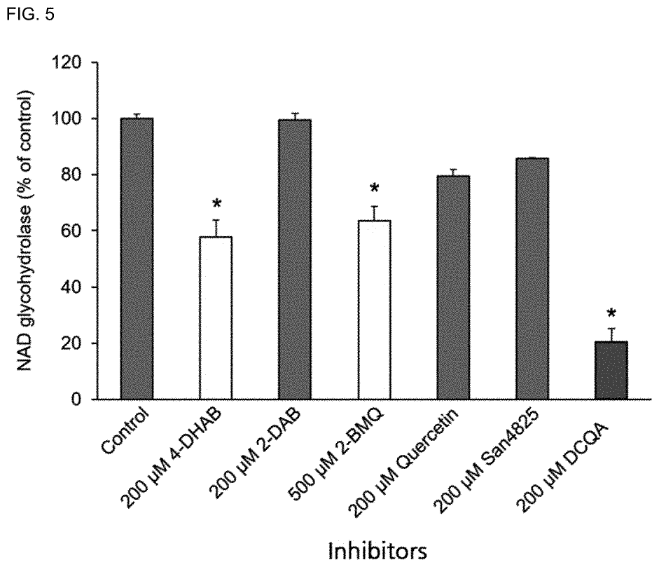

In order to find an inhibitor which inhibits the activation of new ADP-ribosyl cyclase (ADPRC) of SEQ ID NO 1, 5 μL of each of 4,4′-dihydroxyazobenzene (4-DHAB, TCI (Japan)), 2,2′-dihydroxyazobenzene (2-DAB, Sigma-Aldrich (USA)), 2-(3,4-dihydroxyphenyl)-3,5,7-trihydroxychromen-4-one (Quercetin, Sigma-Aldrich (USA)) and San4825 (synthesized by Kannt A et al. [Kannt A, Sicka K, Kroll K, Kadereit D, Gogelein H. Naunyn. Schmiedebergs. Arch. Pharmacol. 385: 717-727, 2012], China) and 2-(1,3-benzoxazol-2-ylamino)-1-methylquinazoline-4(1H)-one (2-BMQ) and 1,4-dicaffeoylquinic acid (1,4-DCQA, Biopurify (China), hereinafter DCQA), which were newly found through screening, was reacted with 40 μL of new ADPRC in iced water for 15 minutes. Then, reaction was conducted at 37° C. for 1 hour after treating with 5 μL of 2 mM ε-NAD (nicotinamide 1,N6-ethenoadenine dinucleotide). Then, the enzyme-substrate reaction was stopped by treating with 50 μL of 10% trichloroacetic acid. After centrifuging for 10 minutes and adding 80 μL of the supernatant to 720 μL of 0.1 M sodium phosphate buffer, absorbance was measured at 297 nm (excitation) and 410 nm (emission) using a fluorescence spectrometer. The result is shown in FIG. 5. As shown in FIG. 5, the NAD glycohydrolase (NADase) activity was inhibited by 42.40% for 4-DHAB, 36.49% for 2-BMQ and even 79.64% for DCQA as compared to the control group.

### Example 5. Effect of DCQA on Blood Glucose, Kidney-to-Body Weight Ratio, Creatinine Clearance Rate and Urine Albumin Level in Renal Disease Mouse Model

The blood glucose, kidney-to-body weight ratio, creatinine clearance rate and urine albumin level in a renal disease mouse model were measured by the method reported by Kim S Y et al. [Kim S Y, Park K H, Gul R, Jang K Y, Kim U H. Am. J. Physiol. Renal Physiol. 296: F291-F297, 2009]

Specifically, 0.2 mL of 50 mM citrate buffer (pH 4.8) (control group and DCQA group) or 200 μL of 5 mg/mL streptozotocin (STZ, Sigma-Aldrich (USA)) dissolved in 50 mM citrate buffer (STZ group and STZ+DCQA group) was intraperitoneally injected to C57BL/6J mice. Blood glucose was measured from day 2 after the STZ injection and the mice whose blood glucose level was 300 mg/dL were divided into an STZ group and an STZ+DCQA group. To the mice that showed increased blood glucose level, DCQA which showed the highest inhibitory effect against novel ADPRC in Example 5 was intraperitoneally administered every day (100 μL) for 6 weeks after dissolving in dimethyl sulfoxide to 9 mg/mL and diluting with saline to 9 μg/mL. On the last day, the mouse was put in a metabolic cage and urine was collected for 24 hours.

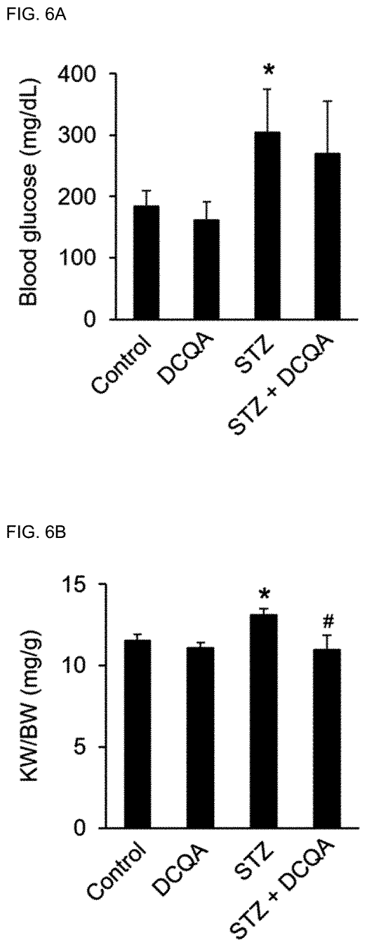

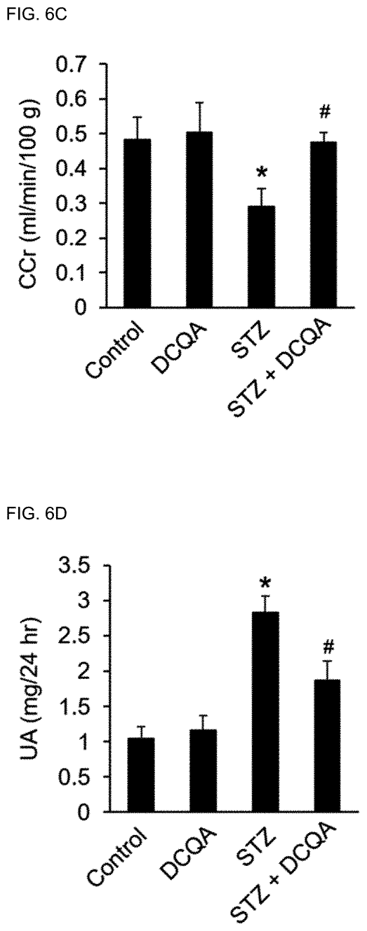

Then, the body weight and blood glucose of the mouse were measured (FIG. 6A). In addition, in order to investigate the possibility as a therapeutic agent for a renal disease, the mouse was sacrificed and serum and kidney were taken. After measuring kidney-to-body weight ratio (FIG. 6B), some of the kidney was fixed in 10% formalin for fluorescent staining and H&E staining, and the remainder was subjected to ADPR cyclase activity and cADPR concentration measurement. Creatinine clearance rate was calculated by measuring the creatinine levels in urine and serum using a creatinine assay kit (Bioassay Systems, USA) (FIG. 6C), and urine albumin level was measured using an albumin assay kit (Bioassay Systems, USA) (FIG. 6D). The result is shown in FIGS. 6A to 6D.

As can be seen from FIGS. 6A to 6D, although the STZ+DCQA group did not show decreased in blood glucose as compared to the control group (STZ) (FIG. 6A), the possibility as a candidate for a therapeutic agent for a renal disease such as chronic renal failure or diabetic nephropathy was confirmed from the significant decrease in kidney to body weight (FIG. 6B), significant increase in creatinine clearance rate (FIG. 6C) and significant decrease in the urine albumin level (FIG. 6D).

### Example 6. Effect of DCQA on ADPRC Activity and cADPR Concentration in Kidney Tissue of Renal Disease Mouse Model

The ADPRC activity and cADPR concentration in the kidney tissue of a renal disease mouse model were measured by the method reported by Kim S Y et al. [Kim S Y, Park K H, Gul R, Jang K Y, Kim U H. Am. J. Physiol. Renal Physiol. 296: F291-F297, 2009]

Specifically, 0.2 mL of 50 mM citrate buffer (pH 4.8) (control group and DCQA group) or 200 μL of 5 mg/mL streptozotocin (STZ, Sigma-Aldrich (USA)) dissolved in 50 mM citrate buffer (STZ group and STZ+DCQA group) was intraperitoneally injected to C57BL/6J mice. Blood glucose was measured from day 2 after the STZ injection and the mice whose blood glucose level was 300 mg/dL were divided into an STZ group and an STZ+DCQA group. To the mice that showed increased blood glucose level, DCQA which showed the highest inhibitory effect against novel ADPRC in Example 5 was intraperitoneally administered every day (100 μL) for 6 weeks after dissolving in dimethyl sulfoxide to 9 mg/mL and diluting with saline to 9 μg/mL. In order to investigate the possibility as a therapeutic agent for a renal disease, the mouse was sacrificed and serum and kidney were taken.

After lysing some of the kidney tissue with a lysis buffer, 45 μL of the lysed sample was treated with 5 μL of 2 mM NGD (nicotinamide guanine dinucleotide) and reacted at 37° C. for 1 hour. Then, the enzyme-substrate reaction was stopped by treating with 50 μL of 10% trichloroacetic acid. After centrifuging for 10 minutes and adding 80 μL of the supernatant to 720 μL of a 0.1 M sodium phosphate buffer, absorbance was measured at 297 nm (excitation) and 410 nm (emission) with a fluorescence spectrometer (Hitachi, Japan).

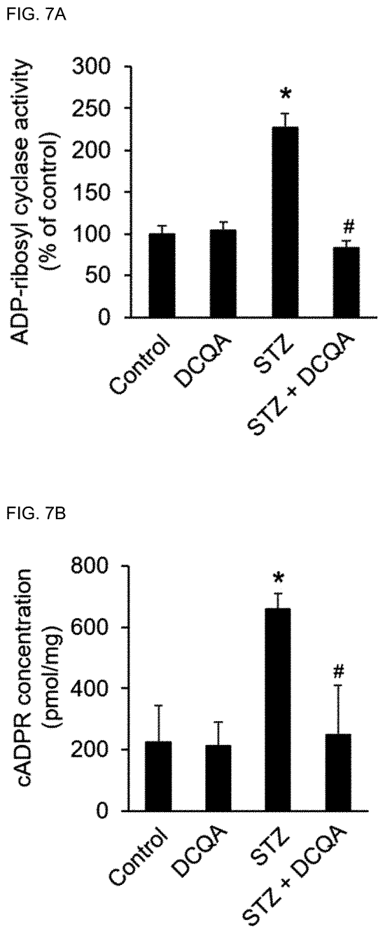

In addition, after extracting cADPR by treating some of the kidney tissue taken from each group with 0.2 mL of 0.6 M trichloroacetic acid, 0.1 mL of the extract or 0.1 mL of a standard cADPR solution was reacted at room temperature for 30 minutes after adding 50 μL of a mixture solution of ADPR cyclase (0.3 μg/mL), nicotinamide (30 mM) and sodium phosphate (100 mM). The mixture solution was reacted for 2-4 hours after adding ethanol (2%), alcohol dehydrogenase (100 μg/mL), resazurin (20 μM), diaphorase (10 μg/mL), FMN (10 μM), nicotinamide (10 mM), bovine serum albumin (BSA, 0.1 mg/mL) and sodium phosphate (100 mM). Then, absorbance was measured between 544 nm and 590 nm using a fluorescence spectrophotometer. The result is shown in FIG. 7.

As shown in FIG. 7A, the DCQA of the present disclosure decreased the ADP-ribosyl cyclase activity increased by STZ to the level of the control group. And, as shown in FIG. 7B, the DCQA of the present disclosure decreased the cADPR concentration increased by STZ to the level of the control group. Therefore, it was confirmed that DCQA has the ability to decrease ADPRC activity and cADPR concentration, which are increased due to failure or loss of kidney function associated with a renal disease.

### Example 7. Effect of 1,4-DCQA on Change in Expression of TGF-β1, Fibronectin and Collagen IV in Kidney Tissue of Renal Disease Mouse Model

The change in the expression of TGF-β1, fibronectin and collagen IV in the kidney tissue of a renal disease mouse model was measured by the method reported by Kim S Y et al. [Kim S Y, Park K H, Gul R, Jang K Y, Kim U H. Am. J. Physiol. Renal Physiol. 296: F291-F297, 2009].

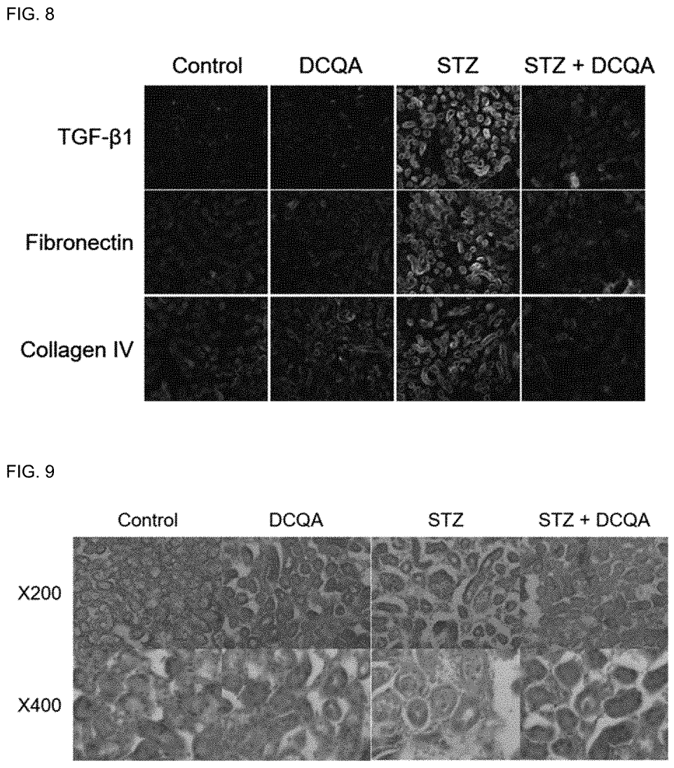

Specifically, 0.2 mL of 50 mM citrate buffer (pH 4.8) (control group and DCQA group) or 200 μL of 5 mg/mL streptozotocin (STZ, Sigma-Aldrich (USA)) dissolved in 50 mM citrate buffer (STZ group and STZ+DCQA group) was intraperitoneally injected to C57BL/6J mice. Blood glucose was measured from day 2 after the STZ injection and the mice whose blood glucose level was 300 mg/dL were divided into an STZ group and an STZ+DCQA group. To the mice that showed increased blood glucose level, DCQA which showed the highest inhibitory effect against novel ADPRC in Example 5 was intraperitoneally administered every day (100 μL) for 6 weeks after dissolving in dimethyl sulfoxide to 9 mg/mL and diluting with saline to 9 μg/mL. In order to investigate the possibility as a therapeutic agent for a renal disease, the mouse was sacrificed and serum and kidney were taken. Some of the extracted kidney was fixed in 10% formalin. The kidney tissue fixed in 10% formalin was cut into kidney tissue sections on a slide glass using a cryostat microtome. The kidney tissue sections were washed with a TTBS (Tris-buffered saline (TBS) with 0.1% Tween 20) buffer and then incubated with a TTBS buffer containing 1% bovine serum albumin (BSA) for 1 hour. The tissues were reacted with primary antibodies (TGF-β1 (Santa Cruz, USA), fibronectin (Santa Cruz, USA) and collagen IV (Abcam, UK)) diluted in a TTBS buffer containing 1% bovine serum albumin (BSA) to 1:200 at 4° C. for 12 hours or longer. After washing the tissues that reacted with the primary antibodies 3 times with a TTBS buffer, they were reacted with FITC-labeled secondary antibodies diluted in a TTBS buffer to 1:200 in the dark at room temperature for 1 hour. Then, after washing 3 times with a TTBS buffer, a cover glass was attached using a mounting solution. The stained kidney tissue was observed using a fluorescence microscope (Carl Zeiss, Germany) for observing green fluorescence. The result is shown in FIG. 8.

As shown in FIG. 8, the DCQA of the present disclosure decreased the expression level of TGF-β1, fibronectin and collagen IV increased by STZ to the level of the control group. Therefore, it was confirmed that DCQA has the ability to decrease the expression level of TGF-β1, fibronectin and collagen IV, which are increased due to failure or loss of kidney function associated with a renal disease.

### Example 8. Investigation of Histopathological Change in Kidney Tissue of Renal Disease Mouse Model Through Hematoxylin and Eosin (H&E) Staining

The histopathological change in the kidney tissue of a renal disease mouse model was measured by the method reported by Shu B et al. [Shu B, Feng Y, Gui Y, Lu Q, Wei W, Xue X, Sun X, He W, Yang J, Dai C. Cell. Signal. 42:249-258, 2018]

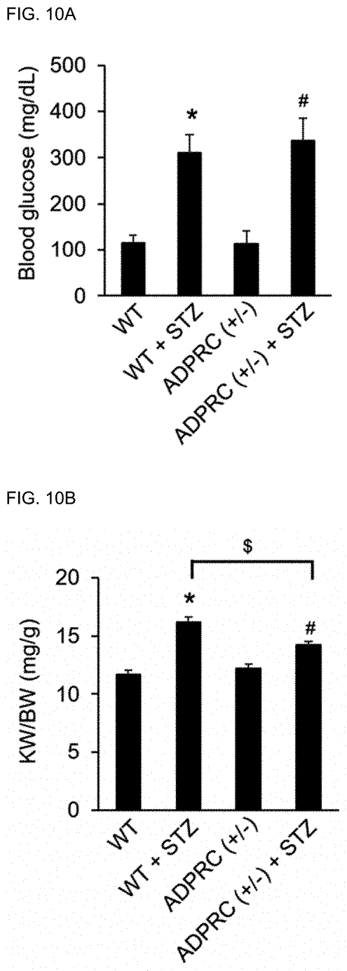

Specifically, 0.2 mL of 50 mM citrate buffer (pH 4.8) (control group and DCQA group) or 200 μL of 5 mg/mL streptozotocin (STZ, Sigma-Aldrich (USA)) dissolved in 50 mM citrate buffer (STZ group and STZ+DCQA group) was intraperitoneally injected to C57BL/6J mice. Blood glucose was measured from day 2 after the STZ injection and the mice whose blood glucose level was 300 mg/dL were divided into an STZ group and an STZ+DCQA group. To the mice that showed increased blood glucose level, DCQA which showed the highest inhibitory effect against novel ADPRC in Example 5 was intraperitoneally administered every day (100 μL) for 6 weeks after dissolving in dimethyl sulfoxide to 9 mg/mL and diluting with saline to 9 μg/mL. In order to investigate the possibility as a therapeutic agent for a renal disease, the mouse was sacrificed and serum and kidney were taken. Some of the extracted kidney was fixed in 10% formalin. The kidney tissue fixed in 10% formalin was cut into kidney tissue sections on a slide glass using a cryostat microtome. The kidney tissue sections were washed with running water for 5 minutes and then stained with hematoxylin for 5 minutes. After the staining, the tissue was washed with running water for 5 minutes. Then, the tissue was immersed in 157 mM hydrochloric acid 2 times and taken out quickly. Then, the tissue was immersed once in 0.25% ammonia water and taken out quickly. After washing again with running water for 5 minutes and staining with eosin for about 30 seconds, the tissue was reacted with 70% ethanol for 30 seconds, with 80% ethanol for 30 seconds, with 90% ethanol for 30 seconds, with 100% ethanol for 30 seconds, again with 100% ethanol for 30 seconds, and then again with 100% ethanol for 30 seconds. Finally, after reacting with xylene for 5 minutes and again with xylene for 5 minutes or longer, a cover glass was attached using a mounting solution. The stained kidney tissue was observed with an optical microscope (Lieca, Germany). The result is shown in FIG. 9.

As shown in FIG. 9, the DCQA of the present disclosure recovered the formation of glomerulus hypertrophy, infiltration of inflammatory cells and formation of transitional epithelial cells increased by STZ to a level similar to that of the control group. Therefore, it was confirmed that DCQA has the ability to recover the histopathological changes of the kidney caused by failure or loss of kidney function associated with a renal disease.

### Example 9. Comparison of Blood Glucose, Kidney-to-Body Weight Ratio and Creatinine Clearance Rate Between Normal Mouse and ADPRC Hetero (ADPRC (+/−)) Mouse Renal Disease Model

The blood glucose, kidney-to-body weight ratio and creatinine clearance rate of a renal disease mouse model were measured by the method reported by Kim S Y et al. [Kim S Y, Park K H, Gul R, Jang K Y, Kim U H. Am. J. Physiol. Renal Physiol. 296: F291-F297, 2009]

Specifically, 200 μL of streptozotocin (STZ) dissolved in a 50 mM citrate buffer (pH 4.8) to 5 mg/mL was intraperitoneally injected to 12951/SvImJ mouse (wild type, WT) and ADPRC hetero knockout (ADPRC(+/−)) mouse acquired from The Jackson Laboratory (USA). Blood glucose was measured from day 2 after the injection and the mice whose blood glucose level was 300 mg/dL were used. 6 weeks later, the mouse was put in a metabolic cage and urine was collected for 24 hours.

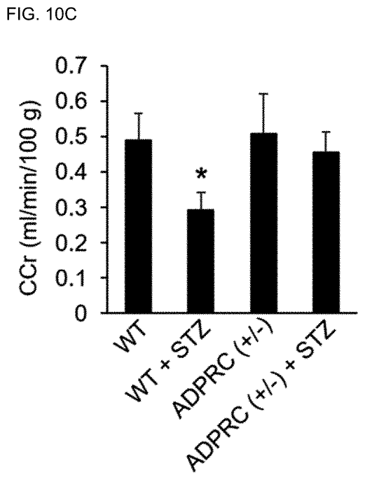

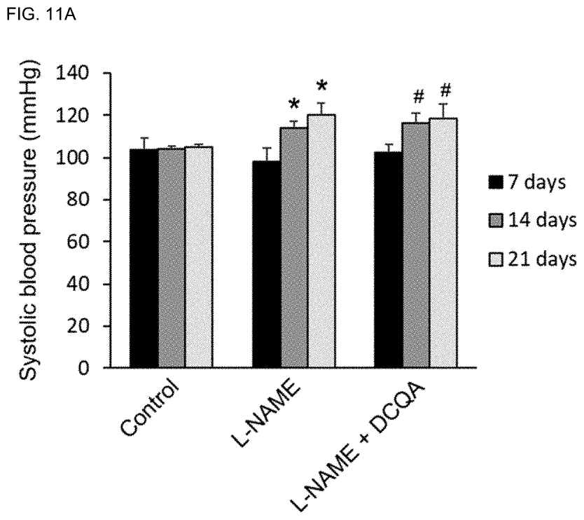

Then, the body weight and blood glucose of the mouse were measured (FIG. 10A). The mouse was sacrificed and serum and kidney were taken. Then, kidney-to-body weight ratio was measured using the kidney (FIG. 10B). Creatinine clearance rate was calculated by measuring the creatinine levels in urine and serum using a creatinine assay kit (Bioassay Systems, USA) (FIG. 10C). The result is shown in FIGS. 10A to 10C.

As shown in FIGS. 10A to 10C, although blood glucose was not decreased in the (ADPRC(+/−) group as compared to the control group (FIG. 10A), significant decrease in kidney to body weight was observed in the ADPRC(+/−)+STZ group of the present disclosure as compared to the WT+STZ group (FIG. 10B). In addition, the decrease in creatinine clearance rate caused by STZ was not observed in the ADPRC(+/−)+STZ group (FIG. 10C), which confirms the importance of the new ADPRC in a renal disease such as chronic renal failure or diabetic nephropathy.

### Example 10. Effect of DCQA on Blood Pressure and Creatinine Clearance Rate in Normal Mouse and Hypertension Mouse Model

The blood pressure and creatinine clearance rate in a hypertension mouse model were measured by the method reported by Allagnat et al. [Allagnat F, Haefliger J A, Lambelet M, Longchamp A, Berard X, Mazzolai L, Corpataux J M, Deglise S. Eur. J. Vasc. Endovasc. Surg. 51: 733-742, 2016] and Kim S Y et al. [Kim S Y, Park K H, Gul R, Jang K Y, Kim U H. Am. J. Physiol. Renal Physiol. 296: F291-F297, 2009]

Specifically, 0.2 mL of 8 mg of L-NAME (Nw-nitro-L-arginine-methyl-ester, Sigma-Aldrich, USA) dissolved in 1 mL of saline was orally administered to C57BL/6J mouse every day. On days 7 and 14 of the oral administration, blood pressure was measured at the tail of the mouse. Then, DCQA which showed the highest inhibitory effect against novel ADPRC in Example 5 was intraperitoneally administered every day (100 μL) for 7 days after dissolving in dimethyl sulfoxide to 9 mg/mL and diluting with saline to 9 μg/mL and L-NAME was administered orally. On day 6 after the DCQA treatment, the mouse was put in a metabolic cage and urine was collected for 24 hours.

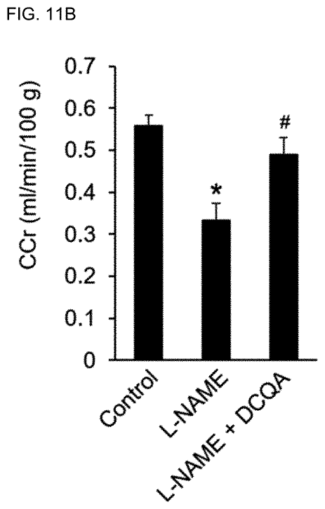

After measuring the blood pressure of the mouse (FIG. 11A), the mouse was sacrificed and serum was obtained.

Creatinine clearance rate was calculated by measuring the creatinine levels in urine and serum using a creatinine assay kit (Bioassay Systems, USA) (FIG. 11B). The result is shown in FIGS. 11A and 11B.

As shown in FIG. 11A, DCQA had no effect of lowering blood pressure in the hypertension model. However, the possibility of DCQA as a therapeutic agent for a renal disease such as hypertensive nephropathy was confirmed through the increase in creatinine clearance rate by DCQA (FIG. 11B).

### REFERENCES

1. Berridge M J, Bootman M D, Roderick H L. Calcium signaling: dynamics, homeostasis and remodeling. Nat Rev Mol Cell Biol. 4: 517-529, 2003.
2. Lee H C. Structure and enzymatic functions of human CD38. Mo/Med. 12: 317-323, 2006.
3. Resnick L M. Ionic basis of hypertension, insulin resistance, vascular disease, and related disorders. The mechanism of “Syndrome X”. Am. J. Hypertens. 6:123S-134S, 1993.
4. Cowley A W Jr. Long-term control of arterial blood pressure. Physiol. Rev. 72: 231-300, 1992.
5. Kim B J, Park K H, Yim C Y, Takasawa S, Okamoto H, Im M J, Kim U H. Generation of nicotinic acid adenine dinucleotide phosphate and cyclic ADP-ribose by glucagon-like peptide-1 evokes Ca²⁺ signal that is essential for insulin secretion in mouse pancreatic islets. Diabetes. 57: 868-878, 2008.
6. Gul R, Park J H, Kim S Y, Jang K Y, Chea J K, Ko J K, Kim U H. Inhibition of ADP-ribosyl cyclase attenuates angiotensin II-induced cardiac hypertrophy. Cardiovasc. Res. 81: 582-591, 2009.
7. Kim S Y, GuI R, Rah S Y, Kim S H, Park S K, Im M J, Kwon H J, Kim U H. Molecular mechanism of ADP-ribosyl cyclase activation in angiotensin II signaling in murine mesangial cells. Am. J. Physiol. Renal. Physiol. 294: F989-F989, 2008.
8. Partida-Sanchez S, Cockayne D A, Monard S, Jacobson E L, Oppenheimer N, Garvy B, Kusser K, Goodrich S, Howard M, Harmsen A, Randall T D, Lund F E. Cyclic ADP-ribose production by CD38 regulates intracellular calcium release, extracellular calcium influx and chemotaxis in neutrophils and is required for bacterial clearance in vivo. Nat Med 7: 1209-1216, 2001.
9. Graeff R, Lee H C. A novel cycling assay for cellular cADP-ribose with nanomolar sensitivity. Biochem. J. 361: 379-384, 2002.
10. Kim S Y, Park K H, GuI R, Jang K Y, Kim U H. Role of kidney ADP-ribosyl cyclase in diabetic nephropathy. Am. J. Physiol. Renal Physiol. 296: F291-F297, 2009.
11. Shu B, Feng Y, Gui Y, Lu Q, Wei W, Xue X, Sun X, He W, Yang J, Dai C. Blockade of CD38 diminishes lipopolysaccharide-induced macrophage classical activation and acute kidney injury involving NF-κB signaling suppression. Cell. Signal. 42: 249-258, 2018.
12. Allagnat F, Haefliger J A, Lambelet M, Longchamp A, Berard X, Mazzolai L, Corpataux J M, Deglise S. Nitric oxide deficit drives intimal hyperplasia in mouse models of hypertension. Eur. J. Vasc. Endovasc. Surg. 51: 733-742, 2016.
13. Kannt A, Sicka K, Kroll K, Kadereit D, Gogelein H. Naunyn. Schmiedebergs. Arch. Pharmacol. 385: 717-727, 2012.

Although the specific exemplary embodiments of the present disclosure have been described in detail, it will be obvious to those having ordinary knowledge in the art that they are merely preferred exemplary embodiments and the scope of the present disclosure is not limited by them. It is to be understood that the substantial scope of the present disclosure is defined by the appended claims and their equivalents.

We claim:

1. An ADP-ribosyl cyclase (ADPRC) comprising an amino acid sequence of SEQ ID NO 1 or a naturally occurring variant thereof.

2. The ADP-ribosyl cyclase or naturally occurring variant thereof according to claim 1, wherein the naturally occurring variant of ADP-ribosyl cyclase is a naturally occurring variant selected from a group consisting of an interspecies variant, a species homolog, an isoform, an allelic variant, a conformational variant, a splice variant and a point mutation variant.

3. The ADP-ribosyl cyclase or naturally occurring variant thereof according to claim 1, wherein the naturally occurring variant of ADP-ribosyl cyclase originates from an organism selected from a group consisting of mammals, birds, reptiles, amphibians and fish.

4. The ADP-ribosyl cyclase or naturally occurring variant thereof according to claim 1, wherein the naturally occurring variant of ADP-ribosyl cyclase is an ADP-ribosyl cyclase comprising an amino acid sequence selected from a group consisting of SEQ ID NOS 2-21.

5. The ADP-ribosyl cyclase or naturally occurring variant thereof according to claim 1, wherein the ADP-ribosyl cyclase or variant thereof converts NAD⁺ to cyclic ADP-ribose (cADPR).

6. A nucleic acid molecule encoding the ADP-ribosyl cyclase or naturally occurring variant thereof according to claim 1.

7. A vector comprising the nucleic acid molecule according to claim 6.

8. A host cell comprising the vector according to claim 7.

9. A method for converting NAD+ into cyclic ADP-ribose (cADPR), comprising the step of treating NAD+ to produce the ADP-ribosyl cyclase or naturally occurring variant thereof according to claim 1, a nucleic acid molecule encoding the ADP-cyclase or naturally occurring variant thereon, or a vector comprising the nucleic acid molecule.

10. A method for preventing or treating an ADP-ribosyl cyclase-mediated disease, comprising administering an inhibitor against the expression or activation of an ADP-ribosyl cyclase comprising an amino acid sequence of SEQ ID NO 1 or a naturally occurring variant thereof as an active ingredient to a subject.

11. The method according to claim 10, wherein the inhibitor against the expression of an ADP-ribosyl cyclase or a naturally occurring variant thereof is selected from a group consisting of an antisense oligonucleotide, a siRNA, a shRNA, a miRNA, a ribozyme, a DNAzyme and a PNA (protein nucleic acid).

12. The method according to claim 11, wherein the siRNA comprises a nucleotide sequence of SEQ ID NO 22.

13. The method according to claim 10, wherein the inhibitor against the activation of the ADP-ribosyl cyclase or naturally occurring variant thereof is selected from a group consisting of a compound, a peptide, a peptide mimetic, an aptamer and an antibody.

14. The method according to claim 13, wherein the compound is selected from a group consisting of 4,4′-dihydroxyazobenzene, 2-(1,3-benzoxazol-2-ylamino)-1-methylquinazolin-4(1H)-one and dicaffeoylquinic acid.

15. The method according to claim 10, wherein the ADP-ribosyl cyclase-mediated disease is a renal disease.

16. The method according to claim 15, wherein the renal disease is renal failure, nephropathy, nephritis, renal fibrosis or nephrosclerosis.

17. The method according to claim 16, wherein the renal failure is chronic renal failure, acute renal failure or mild renal failure before dialysis.

18. The method according to claim 16, wherein the nephropathy is nephropathy syndrome, lipoid nephropathy, diabetic nephropathy, immunoglobulin A (IgA) nephropathy, analgesic nephropathy or hypertensive nephropathy.

19. (canceled)

20. A non-human animal model wherein a hetero-type gene of the ADP-ribosyl cyclase (ADPRC) or the naturally occurring variant thereof according to claim 1 is deleted.

21. A method for identifying an ADP-ribosyl cyclase-mediated disease, comprising:
(a) a step of inducing a specific disease in the animal model according to claim 20 and a wild-type animal model; and
(b) a step of identifying the difference between the animal models.

22. A method for providing information for diagnosis of an ADP-ribosyl cyclase-mediated disease, comprising:
1) a step of measuring the expression or activation level of an ADP-ribosyl cyclase according to claim 1 in a sample isolated from a subject; and
2) a step of determining a risk of the ADP-ribosyl cyclase-mediated disease of the subject by comparing the expression or activation level of the ADP-ribosyl cyclase or naturally occurring variant thereof in the step 1) with a normal control group.

23. A composition for diagnosis of an ADP-ribosyl cyclase-mediated disease, comprising an agent for measuring the gene expression level or protein level of an ADP-ribosyl cyclase according to claim 1.

24. A kit for diagnosis of an ADP-ribosyl cyclase-mediated disease, comprising an agent for measuring the gene expression level or protein level of an ADP-ribosyl cyclase according to claim 1.

25. A method for screening a substance for preventing or treating an ADP-ribosyl cyclase-mediated disease, comprising:
1) a step of treating a cell expressing an ADP-ribosyl cyclase according to claim 1 with a test substance;
2) a step of measuring the gene expression level or protein level of the ADP-ribosyl cyclase as a result of treating with the test substance; and
3) a step of screening the test substance as a substance for preventing or treating an ADP-ribosyl cyclase-mediated disease if the gene expression level or protein level is decreased as compared to a control group not treated with the test substance.
